(19) United States
(12) Patent Application Publication
(10) Pub. No.: US 2011/0301632 A1
(43) Pub. Date: Dec. 8, 2011

(54) ANAL PROLAPSE PREVENTION AID

(75) Inventor: Arata KUSANO, Kita Adati-gun (JP)

(73) Assignee: Kyoto Kogyo Co., Ltd., Kita Adati-gun (JP)

(21) Appl. No.: 13/154,959

(22) Filed: Jun. 7, 2011

(30) Foreign Application Priority Data
Jun. 7, 2010 (JP) 2010-129737
May 24, 2011 (JP) 2011-116234

(51) Int. Cl. A61M 29/00

(52) U.S. Cl. 606/197

(57) ABSTRACT

An anal prolapse prevention aid is configured so as to enable a procedure wherein a stuffing is set inside a cylinder film having the closed end, and the tip of an axle of an insertion stick is fitted into a junction hole in the rear end of a stuffing to thereby be inserted into the anus, and then only the insertion stick is pulled out.

### FIELD OF THE INVENTION

The present invention relates to an anal prolapse prevention aid which returns an external hemorrhoid (verrucose hemorrhoid) accompanied with an internal hemorrhoid prolapsing through the anus back into the anus to thereby relieve pain associated with the prolapse of hemorrhoids, thereby maintaining the therapeutic effect of the hemorrhoid.

### BACKGROUND OF THE INVENTION

The present inventors have already proposed “The anal prolapse prevention aid” through the Patent Document 1 described below. As illustrated in FIG. 14, this aid includes a flexible and rigidity-free cylindrical film which has the one opened end and another closed end to form a longer balloon than a determined one, and the closed end of the film, into which a stuffing is inserted, is tied to forma neck, through which the film is then extended toward the opposite side to a head as far as it reaches outside the anus.

There is a dentate line between the rectal intestine and the anus at the lower end of the rectal intestine, and the rectal intestine side of the dentate line has a so-called cushion. Furthermore, their outer circumferential surfaces are surrounded by an internal sphincter muscle and an outer sphincter muscle. An external hemorrhoid (verrucose hemorrhoid) accompanied with an internal hemorrhoid often projects itself in the cushion. These hemorrhoids place various kinds of burdens on the anus in one's daily life, to thereby loosen or cleave the tissue supporting the cushion of the rectal intestine, resulting in the enhanced congestion of the cushion.

Then, when the internal hemorrhoid gradually gets larger, it slips out the anus during defecation and then naturally returns back into the anus. But the internal hemorrhoid, which gets much larger to thereby step up into a state as illustrated in FIG. 15, is left to slip out of the anus, and thus can not return back without a finger pushing it in. This state is referred to as anal prolapse.

Such anal prolapse is tightened up on the upper side by the involuntary muscle of the anus. The internal hemorrhoid, which is left to slip out, worsens the lesion to cause the progression of hemorrhoid (Cited from “How to Live with Hemorrhoids Skillfully”, written by Jyun'ichi Iwatare, published by Kodansha KK).

### DESCRIPTION OF THE RELATED ART

The anal prolapse prevention aid by the present inventors as described in JP No. 3951209 is used for a procedure wherein, after a hemorrhoid is pushed in by a finger, the forefinger or the like is inserted into the film of the anal prolapse prevention aid as illustrated in FIG. 13, and then pushes the aid in the anus to retain the internal hemorrhoid with the aid of the stuffing present in the tip of the aid rubber balloon, followed by pulling out the finger to retain the stuffing in the anus, thereby preventing anal prolapse.

The conventional anal prolapse prevention aid often causes an error that, after a finger inserts the aid into the anus, the finger is pulled out to thereby cause the simultaneous drawing-back of the aid.

Because the involuntary muscle in the anus works to tighten both the inserted finger and the balloon film, the film prolapses outside together with the pulled-out finger.

Thus, the present inventors have made various kinds of experimental studies, and found out a condition under which the film is exempted from returning-back to accompany the pulling-out of the finger even if the involuntary muscle works, thereby having completed the present invention.

### SUMMARY OF THE INVENTION

The present invention according to a first aspect thereof is an anal prolapse prevention aid comprises:
a cylinder film (1) made up of a flexible and rigidity-free cylindrical film which has one opened end and another closed end;
a stuffing (3) which is to be disposed in the closed end of the cylinder film (1) and has a junction hole (3a) in the rear end; and
an insertion stick (6) which has the one end side of an axle (4) having a small radius to be fitted removably into the junction hole (3a) and another end side of a grip (5) having a larger radius than that of the axle (4), and which is configured so as to enable a procedure wherein the tip of the axle (4) is fitted into the junction hole (3a) of the stuffing (3) set in the cylinder film (1)to insert them into the anus and then retain them inside the anus when the insertion stick is pulled out of the junction hole (3a).

The present invention according to a second aspect thereof is the anal prolapse prevention aid according to the first aspect thereof, wherein

the cylinder film (1) includes the front end of a small diametric compartment (1a) having a small diameter, the rear end of a large diametric compartment (1b), and a tapering compartment (1c) connecting the former two compartments, the stuffing (3) being inserted into the small diametric compartment (1a).

The present invention according to a third aspect thereof is the anal prolapse prevention aid according to the first or second aspect thereof, wherein

the stuffing (3) has rubber-like elasticity, is retained by the tip of the axle (4) of the insertion stick (6) fitted into the junction hole (3a), the outer circumferential surface of the tip of the stuffing (3a) being chamferred so as to become smooth, the outer circumferential surface being furnished with a plurality of degassing gutters (3b) along the axial line.

The present invention according to a fourth aspect thereof is an anal prolapse prevention aid comprising:

a main body (12) which has a head (11) and a cylinder film compartment (1e) integrated together with each other, wherein the compartment is made up of a flexible and rigidity-free cylinder film along the circle of the rear end of the head (11), the head (11) being provided with a conjunction hole (3a) in the center of the rear end; and an insertion stick (6) which has the one end side of an axle (4) having a small radius to be fitted removably into the junction hole (3a) and another end side of a grip (5) having a larger radius than that of the axle (4), and which is configured so as to enable a procedure wherein the tip of the axle (4) is fitted into the junction hole (3a) of the head (11) set in the cylinder film compartment (1e) to insert them into the anus and then retain them inside the anus when the insertion stick is pulled out of the junction hole (3a).

The present invention according to a fifth aspect thereof is the anal prolapse prevention aid according to the fourth aspect thereof, wherein the head (11) has rubber-like elasticity, is retained by the tip of the axle (4) of the insertion stick (6) fitted into the junction hole (3a), the outer circumferential surface of the tip of the head (11) being chamferred so as to become smooth, the outer circumferential surface being furnished with a plurality of degassing gutters (3b) along the axial line.

The present invention according to a sixth aspect thereof is the anal prolapse prevention aid according to the first or fourth aspect thereof, wherein

the axle (4) is supported by the cylindrical grip (5) through a compression spring (7) in the insertion stick (6) so as to move freely forward and backward, and is biased such that the axle (4) advances forward while shifting backward, even when a pressure is applied to the tip of the axis (4) in the direction toward the rear of the axis (4).

The present invention according to the seventh aspect thereof is the anal prolapse prevention aid according to the first or fourth aspect thereof, wherein

the axle (4) of the insertion stick (6) has a diameter of 2 mm to 4 mm.

The present invention according to an eighth aspect thereof is the anal prolapse prevention aid according to any of the first to seventh aspects thereof, wherein

the outer circumferential surface of the tip of the axle (4) of the insertion stick (6) is chamferred so as to become smooth.

The anal prolapse prevention aid of the present invention according to the first and fourth aspects thereof has a cylinder film 1 or a cylinder film compartment 1e (hereinafter referred to as a cylinder film 1 or the like.), a stuffing 3 or a head 11 (hereinafter referred to as a stuffing 3 or the like.) located at the end of the cylinder film 1 or the like, and an insertion stick 6, and is configured so as to enable a procedure wherein the tip of the axle 4 of the insertion stick 6 is fitted into the junction hole 3a of the stuffing 3 or the like. to insert them into the anus and then retain them inside the anus when the insertion stick 6 is pulled out the junction hole 3a.

Therefore, this aid allows the safe and easy retention, in the anus, of the cylinder film 1 or the like containing the stuffing 3 or the like in the tip.

When the stuffing 3 or the like, covered with the cylinder film 1 or the like is inserted into the anus, there is no possibility of the whole of the cylinder film 1 or the like being drawn out of the anus even if the insertion stick 6 is drawn back, because the axle 4 of the insertion stick 6 has a small diameter.

Additionally, because the insertion stick 6 having the axle 4 performs the insertion into the anus, there is no possibility of the inside of the anus being wounded, and the cylinder film 1 or the like containing the stuffing 3 or the like can be safely inserted into the anus.

In the second aspect of the invention, the cylinder film, which is formed so as to have the front end of a small diametric compartment 1a and the rear end of a large diametric compartment 1b, and which retains securely the stuffing 3 inside the tip to facilitate the insertion when the central part of a tapering compartment 1c connects the former two compartments, and thus can provide the anal prolapse prevention aid having a presentable appearance.

In the third and fifth aspects of the invention, the stuffing 3 or the like has rubber-like elasticity and the tip of the axle 4 of the insertion stick 6 is fitted into the junction hole 3 to be retained therein, allowing the safer retention in the anus of the anal prolapse prevention aid.

Additionally, a plurality of degassing gutters 3b has been formed in the outer circumferential surface of the stuffing 3 or the like along the axial line, facilitating flatus and handling in a state where the anal prolapse prevention aid is kept to be inserted into the anus.

In the sixth aspect of the invention, the grip 5 of the insertion stick 6 retains the axle 4 so as to move freely forward and backward through the compression spring 7, causing little pain in the inflammatory lesion of a hemorrhoid when the cylinder film 1 is inserted together with the covered stuffing 3 into the anus.

Since the axle 4 is biased such that the axle 4 advances forward while shifting backward, even when a pressure is applied, at the time of the insertion, to the tip of the axis 4 in the direction toward the rear of the axis, an inflamed anus can be prevented from being applied to a violently strong pressure.

In the seventh aspect of the invention, the axle 4 of the insertion stick 6, which has a diameter of 2 to 4 mm, can be securely pulled out of the anus without giving a feeling of discomfort in the anus.

According to an experiment, the axle 4 of the insertion stick 6, which has a diameter of more than 4 mm, is inserted to thereby be tightened by the action of the involuntary muscle of the anus in the outer circumferential surface, and thus is pulled out of the anus. Therefore, there is a possibility of the cylinder film 1 simultaneously being drawn out. When the axle 4 of the insertion stick 6 has a diameter of 2 mm or less, there is a possibility of lack of stability at the time of insertion.

Further, when the outer circumferential surfaces around the tip of the stuffing 3 and the tip of the axle 4 of the insertion stick 6 are chamferred so as to become smooth, a safer anal prolapse prevention aid is provided.

### BRIEF DESCRIPTION OF THE DRAWINGS

FIG. 1 shows a perspective view of the anal prolapse prevention aid according to the present invention.

FIG. 2 shows a plan view and an elevation view of the cylinder film 1 in the same aid.

FIG. 3 shows an elevation view, a bottom view, and a C-C sectional view of the stuffing 3 in the same aid.

FIG. 4 shows an explanatory diagram of the anal prolapse prevention aid when it is inserted.

FIG. 5 shows a partial longitudinal sectional view of the insertion stick in the same aid.

FIG. 6 shows a perspective view of the decomposed insertion stick 6, and a perspective view of the cylinder film 1, in the same aid.

FIG. 7 shows an explanatory diagram of the situations of the anal prolapse prevention aid when and after it is used.

FIG. 8 shows a perspective view of the main boy in another anal prolapse prevention aid according to the present invention.

FIG. 9 shows an elevation view of the same main body.

FIG. 10 shows a plan view of the same main body.

FIG. 11 shows an X-X longitudinal sectional view of FIG. 9.

FIG. 12 shows an explanatory diagram of the same anal prolapse prevention aid when it is used.

FIG. 13 shows an explanatory diagram of a conventional anal prolapse prevention aid when it is used.

FIG. 14 shows a longitudinal sectional view of the same anal prolapse prevention aid.

FIG. 15 shows an explanatory diagram of the inflammation lesion of the anus in anal prolapse.

### DESCRIPTION OF THE EMBODIMENTS

Referring to Figures, the embodiments of the present invention is explained below.

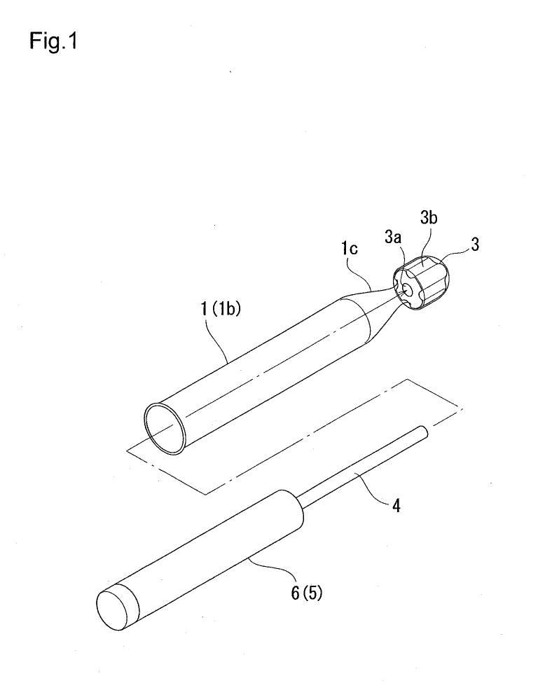

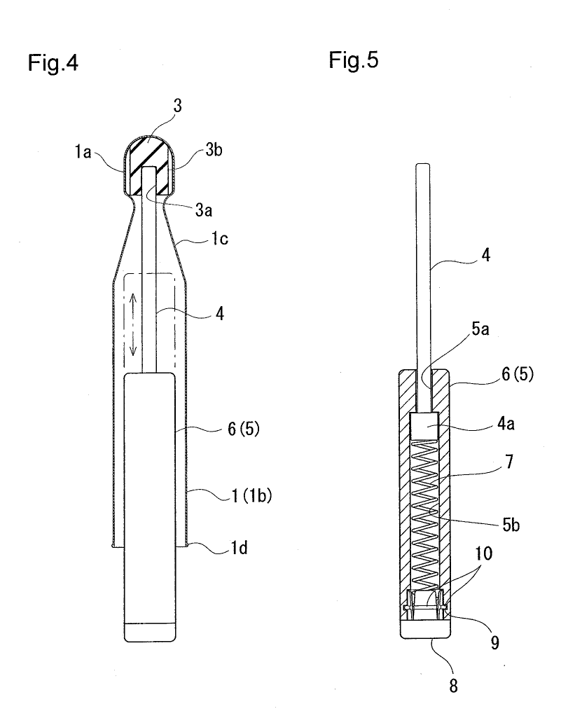

FIG. 1 and FIG. 4 show the embodiments of the anal prolapse prevention aids of the present invention, and they include each a cylinder film 1, a stuffing 3, and an insertion stick 6.

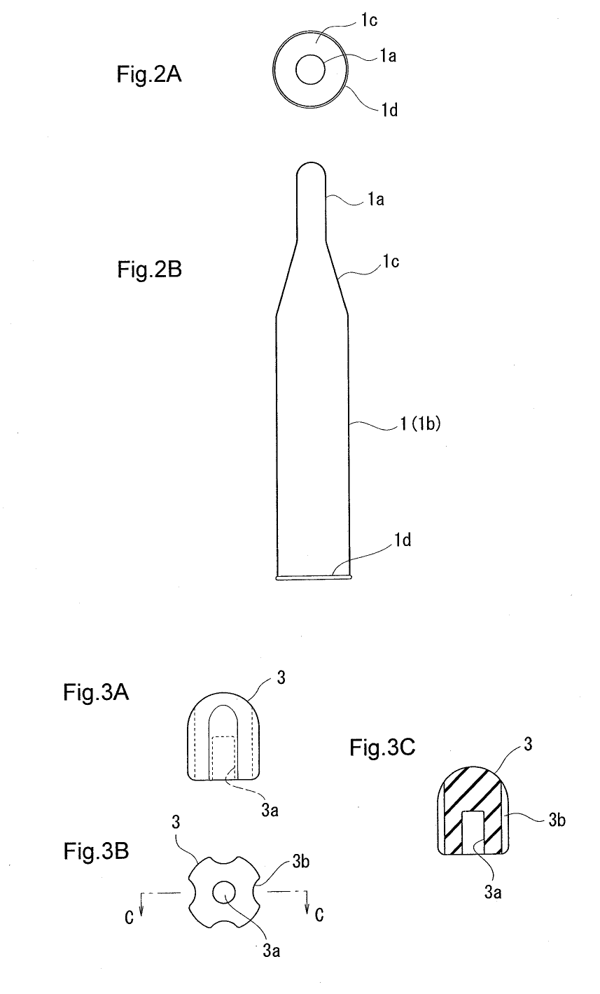

As illustrated in FIG. 2, the cylinder film 1 has the one opened end 1d and another closed end. Furthermore, the film has the rear end of a large diametric compartment 1b, the front end of a small diametric compartment 1a, and the center part of a tapering compartment 1c. The cylinder film 1 is made up of a flexible and rigidity-free cylindrical film, and can be made by using polyurethane and latex. Polyurethane is preferable to use in order to save from rubber allergy. A transparent or translucent color is preferable to be used. These materials are very expandable and elastic. These can have a rubber hardness of 70 to 75 Hs JIS A and has preferably a thickness of 1 mm or less, more preferably 0.1 mm or less.

Moreover, they may have the same material quality, elongation and thickness as a condom. Then, the opened end 1d is formed to be a ring. It should be noted that the cylinder film 1 has a scale, for example, a whole length of about 100 mm, and the small diametric compartment 1a has a length of about 20 mm, while the small diametric compartment 1a and the tapering compartment have a total length of about 30 mm. The small diametric compartment 1a has a free diameter of about 6 mm, while the tapering compartment 1c has a free diameter of about 18 mm. The product has preferably a thickness of 0.03 to 0.04 mm.

Next, as illustrated in FIG. 3, the stuffing 3, which is to be inserted in the closed end of the small diametric compartment 1a of the cylinder film 1, is formed to have a slippery hemispheric tip, on the outer circumferential surface of which the degassing gutters are formed along the axial line to have an angle of 90° between each other in this embodiment. Then, on the bottom of the stuffing 3 is made the junction hole 3a, which has a depth as far as the middle of the stuffing 3. The junction hole 3a has substantially the same outer diameter as the axle 4 of the insertion stick 6, as will explained later.

The flexible silicone rubber molded product, for example, can be used as a material quality for the stuffing 3. Instead of the silicone, the elastomer such as polyurethane and a block copolymer of ESBS (styrene/ethylene/butylene/styrene) also can be used. That is, the material quality is a polymer which has a rubber-like elasticity at room temperature, is elongated at room temperature to thereby have a doubled length or more, and then returns back instantly to the original shape when an external force is eliminated. The polymer has preferably a rubber-like elasticity of 15 A to 35 A (15 to 35 points by the A type tester). The stuffing 3 has a larger outer diameter than that of the small diametric component 1a of the cylinder film 1, and when the stuffing is set in the small diametric component 1a of the cylinder film 1, as shown in FIG. 1 and FIG. 4, the small diametric component 1a is expanded into the same shape as the stuffing 3 in terms of outer circumferential surface. Then, the small diametric component 1a gets a bundled basement. Meanwhile, for example, the stuffing 3 has an outer diameter of about 14 mm and a length of about 15 mm. The tip of the stuffing 3 has a curvature radius of about 7 mm, while the junction hole has a diameter of about 3.5 mm and a depth of about 8 mm. The degassing gutter 3b may have a radius of about 2 mm.

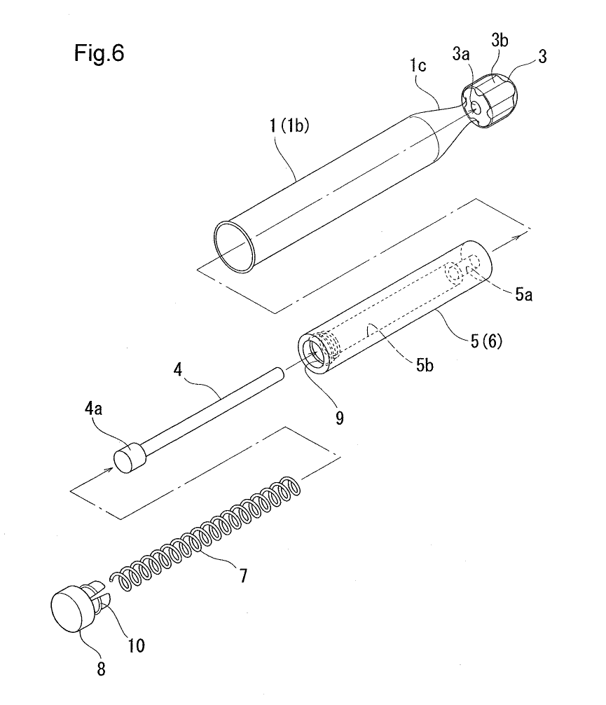

As illustrated in FIG. 5 and FIG. 6, the insertion stick 6 comprises a grip 5, an axle 4, a compression spring 7, and an end cap 8.

The grip 5, which is formed to be cylindrical, has the small diametric component 5a formed in the tip, the large diametric component 5b formed in the rear, and an annular groove 9 set on the inside surface of the rear end. The axle 4 is formed to be integrated with an expansion 4a in the rear end. Duracon can be used as a material quality for the insertion stick 6.

The axle 4 and the grip 5 have each a length of about 60 mm, for example. The large diametric hole 5b has a length of about 50 mm. The compression spring to be inserted into the large diametric hole 5b has a free length of about 60 mm, is made of a coil spring, and is compressed to set inside the grip 5.

Then, the grip 5 is furnished with the axle 4, the compression spring 7, and the end cap 8 according to this order. The annular convex portion 10 is engaged with the annular groove 9 to reach a state illustrated in FIG. 5. The compression spring 7 biases the axle 4 toward the tip side of the axis. The bias is relatively weak to the extent that when a pressure is applied to the tip of the axle 4 rearwards, the axle 4 can easily shift toward the grip 5 side.

(Action)

How to use the anal prolapse prevention aid will be explained below.

First, as illustrated in FIG. 4, the stuffing 3 is inserted into the small diametric compartment 1a of the cylinder film 1, and then the tip of the axle 4 of the insertion stick 6 is fitted into the junction hole 3a of the stuffing 3. Next, the lesion of anal prolapse is returned back inside the anus by a finger. The anal prolapse prevention aid, which is previously assembled into the configuration as illustrated in FIG. 4, is gripped via the grip 5, and then the stuffing 3 is inserted inside through the anus together with the cylinder film 1. The action of the involuntary muscle of the anus applies a pressure to the stuffing 3 toward the rear. Then, the axle 4 shifts toward the rear against the compression spring 7 and the shock is reduced.

Next, the elastic force of the compression spring 7 is used to insert the stuffing 3 into the anus without stimulating the anus. The grip 5 is inserted as far as the surface of its upper end reaches the entrance of the anus, and then is slightly rolled along a circle to be pulled out. At this time, the axle 4 has so small a diameter that only the insertion stick can be easily pulled out, even if the involuntary muscle of the anus tightens the axle. According to an experiment, if the axle 4 has a diameter of 4 mm or more, because of the action of the involuntary muscle of the anus, simultaneously when the insertion stick 6 is pulled out, the cylinder film 1 tends to be pulled out. When the axle 4 has a diameter of 2 mm or less, it has been found that the axle is fragile in strength, thereby lacking in stability at the time of insertion.

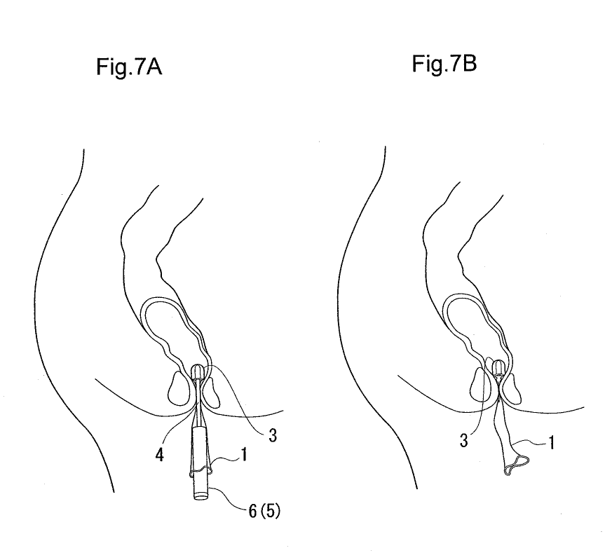

FIG. 7 (A) shows one example of a state of insertion, and then the insertion stick 6 is pulled out to be in a state as illustrated in FIG. 7 (B). In this state, it is only necessary for the open end of the cylinder film 1 to be able to be slightly drawn out without the stuffing 3 slipping out. Therefore, the anal prolapse lesion is retained inside the anus, and is prevented from naturally prolapsing outside.

Additionally, the degassing gutters 3b formed on the outer circumferential surface of the stuffing 3 facilitate an intestinal gas to be discharged. The gas is discharged outside through the gutters.

The open end of the cylinder film 1 may be strongly drawn outwards in order to remove the anal prolapse prevention aid out.

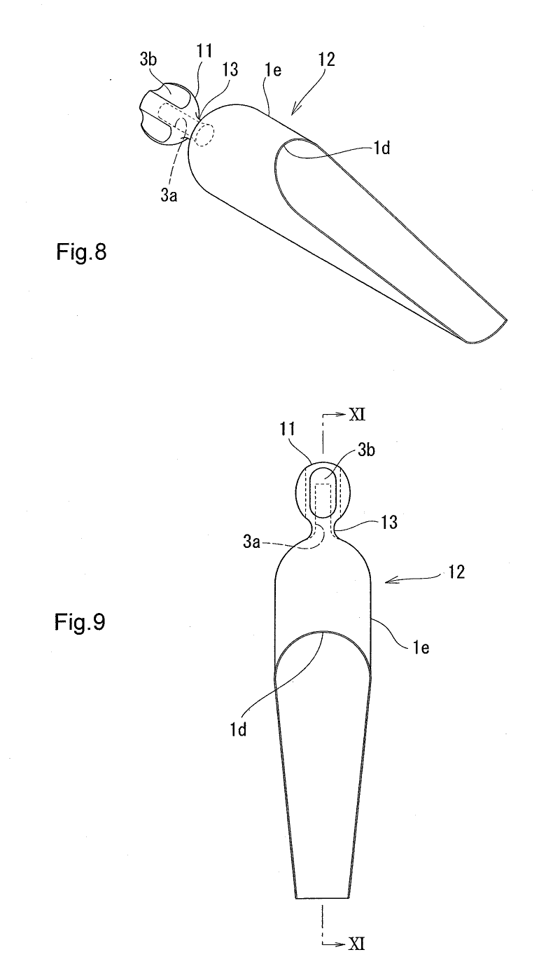

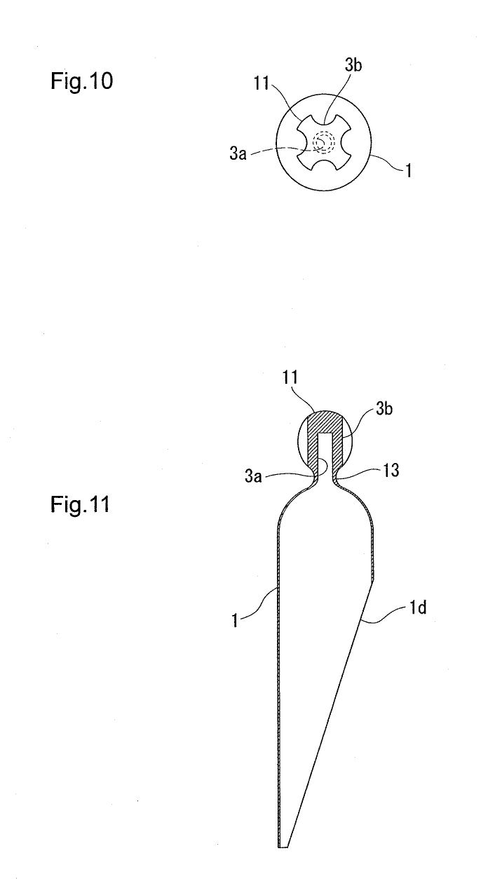

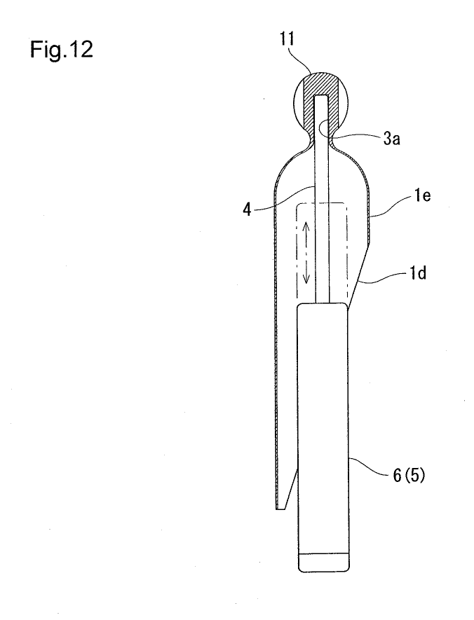

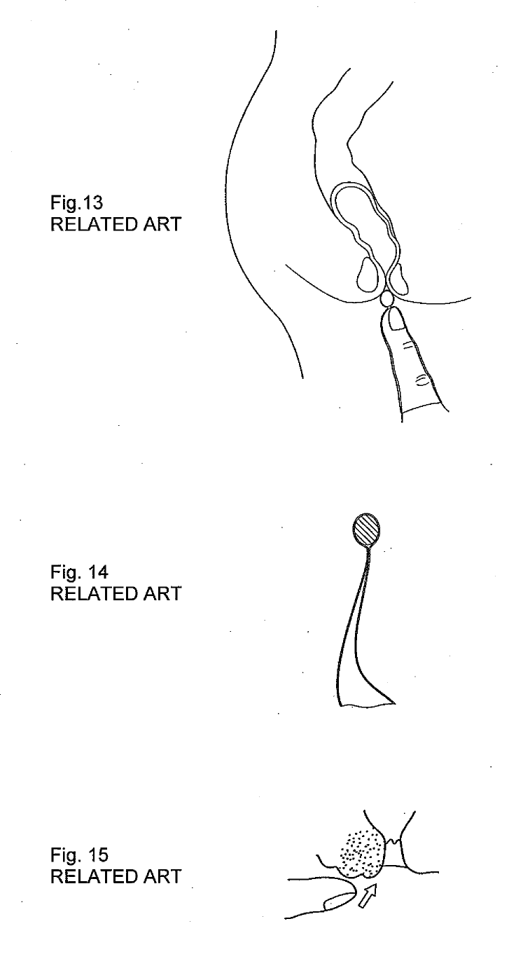

FIG. 8 to FIG. 11 show another embodiment of the anal prolapse prevention aid of the present invention, that is, the main body 12. FIG. 12 shows a situation for using the aid.

What makes the main body 12 of this aid different from the above embodiment is that the latter has the cylinder film 1 and the stuffing 3 separated from each other, while the former has the head 11 and the cylinder film compartment 1e integrated with each other, wherein the head 11 corresponds to the stuffing 3 of the above embodiment and the cylinder film compartment 1e corresponds to the cylinder film 1. That is, the cylinder film compartment 1e and the head 11 are integrally molded (by injection molding) using a same material. A medical urethane rubber or a silicone rubber can be used for the above two.

Then, the main body 12 has the head 11 and the cylinder film compartment 1e integrated with each other, wherein the former is at the front end of main body 12 while the latter is made up of the flexible and rigidity-free cylinder film via the neck 13 along the circle of the rear end of the head 11. At the center of the rear end of the head 11 is formed the junction hole 3a. The cylinder film compartment 1e has an opened end 1d having such a shape as is given by cutting the cylinder slantwise from the middle to the rear end. The shape facilitates a finger to be inserted through the opened end 1d into the cylinder film compartment 1e, when the insertion stick 6 is lost to be used. Furthermore, the shape improves the flexibility of the compartment.

The cylinder film compartment 1e can have a thickness of 0.1 mm to 0.3 mm, preferably 0.15 mm to 0.2 mm. After the head 11 is inserted into the anus as done in the above embodiment, the cylinder film compartment 1e, which is flexible and thin, can be guided outwards as a string to be exposed outside the anus.

This embodiment also uses the insertion stick 6 in the same way as the above embodiment does, and the axle 4 is inserted into the junction hole 3a of the head 11.

1. An anal prolapse prevention aid comprising:
a cylinder film made up of a flexible and rigidity-free cylindrical film which has one opened end and another closed end;
a stuffing which is to be disposed in the closed end of the cylinder film and has a junction hole in the rear end; and
an insertion stick which has the one end side of an axle having a small radius to be fitted removably into said junction hole and another end side of a grip having a larger radius than that of the axle, and which is configured so as to enable a procedure wherein the tip of said axle is fitted into said junction hole of said stuffing set in said cylinder film to insert them into the anus and then retain them inside the anus when the insertion stick is pulled out of said junction hole.

2. The anal prolapse prevention aid according to claim 1, wherein
said cylinder film includes the front end of a small diametric compartment having a small diameter, the rear end of a large diametric compartment, and a tapering compartment connecting the preceding two compartments, said stuffing being inserted into the small diametric compartment.

3. The anal prolapse prevention aid according to claim 1 or 2, wherein
said stuffing has rubber-like elasticity, is retained by the tip of the axle of the insertion stick fitted into the junction hole, the outer circumferential surface of the tip of the stuffing being chamferred so as to become smooth, the outer circumferential surface being furnished with a plurality of degassing gutters along the axial line.

4. An anal prolapse prevention aid comprising:
a main body which has a head and a cylinder film compartment integrated together with each other, wherein the compartment is made up of a flexible and rigidity-free cylinder film along the circle of the rear end of the head, the head being provided with a conjunction hole in the center of the rear end; and
an insertion stick which has the one end side of an axle having a small radius to be fitted removably into said junction hole and another end side of a grip having a larger radius than that of the axle, and which is configured so as to enable a process wherein the tip of said axle is fitted into said junction hole of said head set in said cylinder film compartment to insert them into the anus and then retain them inside the anus when the insertion stick is pulled out of said junction hole.

5. The anal prolapse prevention aid according to claim 4, wherein
said head has rubber-like elasticity, is retained by the tip of the axle of the insertion stick fitted into the junction hole, the outer circumferential surface of the tip of the head being chamferred so as to become smooth, the outer circumferential surface being furnished with a plurality of degassing gutters along the axial line.

6. The anal prolapse prevention aid according to claim 1 or 4, wherein
said axle is supported by said cylindrical grip through a compression spring in said insertion stick so as to move freely forward and backward, and is biased such that the axle advances forward while shifting backward, when a pressure is applied to the tip of the axis in the direction toward the rear of the axis.

7. The anal prolapse prevention aid according to claim 1 or 4, wherein
the axle of said insertion stick has a diameter of 2 mm to 4 mm.

8. The anal prolapse prevention aid according to any of claim 1 or 4,
wherein
an outer circumferential surface of the tip of the axle of the insertion stick is chamferred so as to become smooth.
